(19) United States
(12) Patent Application Publication
(10) Pub. No.: US 2002/0026161 A1
(43) Pub. Date: Feb. 28, 2002

(54) Device and method for managing urine release in incontinent females

(76) Inventors: Reinhold Grundke, Burghausen (DE); Bernhard Liedl, Munchen (DE)

Correspondence Address: BAKER BOTTS LLP, C/O INTELLECTUAL PROPERTY DEPARTMENT, THE WARNER, SUITE 1300, 1299 PENNSYLVANIA AVE, NW, WASHINGTON, DC, 20004-2400, US

(21) Appl. No.: 09/792,054

(22) Filed: Feb. 26, 2001

Related U.S. Application Data
(60) Provisional application No. 60/184,824, filed on Feb. 24, 2000.

(51) Int. Cl. A61M 1/00; A61F 5/44

(52) U.S. Cl. 604/327; 604/355; 600/574

(57) ABSTRACT

The present invention relates to a device and method for managing uncontrolled urine release in females. The device is flexible and is worn externally on the vaginal area. This external device may further be attached to a urine collector, such as a bladder bag, and may be worn by a female person while standing, walking, sitting, or laying down. This external urine drainage device is comfortable and easy to use, avoids soiling of the laundry when worn correctly, runs a low risk of causing urinary infections or skin irritations, and is easily washable and re-usable.

### FIELD OF THE INVENTION

The invention concerns a device for the drainage of uncontrolled urine release in females.

### BACKGROUND OF THE INVENTION

Lack of urinary control (incontinence) is an ailment which affects a large number of adults in varying degrees. Solutions to managing uncontrolled urination, however, are limited. For some people, certain prescription medications assist in bodily control of urine release. In cases where medications do not work, are impracticable, or are only partially successful at controlling urine release, either a catheter is inserted into the bladder to drain the urine into a collection receptacle, or the incontinent person is diapered.

Both catheter use and diapering have disadvantages. The insertion and removal of a catheter requires skill and is most commonly performed by a medical practitioner, requiring a person to remain in a hospital, nursing home, or other managed care setting. Catheters have to be removed and replaced approximately every five days resulting in high costs for people who rely on permanent assistance with urine drainage. Catheters are uncomfortable and the continuous insertion and removal of catheters irritates the urethra and may cause strictures and abscesses to form. Furthermore, catheters run a risk of causing urinary tract and bladder infections. Such infections are dangerous and can even become life threatening in an aging or feeble person.

Diapers, on the other hand, require changing approximately seven times per day also resulting in high costs. Many diapers can be seen through clothing, are bulky and uncomfortable for the wearer, and have the potential for leakage thereby soiling the wearer's clothing. Furthermore, because urine is alkaline, continuous contact between the delicate skin of the genital area and a urine soaked diaper leads to various rashes, skin maceration, and in extreme cases, formation of ulcers.

A need, therefore, exists for a new option for managing uncontrolled urine release which is inexpensive, runs a low risk of causing infections or skin irritations, and is comfortable for a person to use.

### SUMMARY OF THE INVENTION

A device for the drainage of urine release in incontinent females is disclosed. The device comprises a hollow body, a drainage spout, a rim, and a fastening body, wherein the device is worn externally of the female's body. A method for using this device is also disclosed.

The hollow body of the device may comprise a floor section, two side walls, a front wall, and a rear wall, wherein the two side walls, the front wall, and the rear wall are integrally connected with the floor section. The fastening body of the device may comprise a flange such that the rim is shaped to encompass the female's vaginal area and the flange protrudes outwardly from the rim. The fastening body of the device may further comprise a flap attached to the front wall, and a flap attached to the rear wall, each flap having a distal end comprising a fastening element. Methods for using any of these devices are also disclosed.

An advantage of the of the present invention is that it provides an external urine drainage device for females which is comfortable and easy to use, avoids soiling of laundry when applied correctly, runs a low risk of causing urinary infections or skin irritation, provides the wearer with a large range of mobility, and is re-usable.

The urine drainage device for females of the present invention is made from a flexible material in the shape of a hollow body. The hollow body comprises a drainage spout at its distal end and a rim designed to encompass the vagina at its proximal end. The device further comprises a fastening body at its proximal end to connect the device to a holder, such as a hip belt, the waist line of a pair of pants, skirt, tights, underwear, or any other object which will hold the device appropriately in place when worn by a patient. Alternatively, the fastening body may be designed so that it functions as a holder on its own.

When the device is worn, the device is guided between the thighs to mold it against the pelvic bone and to encompass the vagina, and the fastening body is attached to a holder or is worn directly by the wearer. The drainage spout is connected to a bladder bag or other appropriate urine collector either directly or through a flexible tube. Once the device is on the wearer, it can be worn not only while bedridden, but also while standing, walking, or sitting.

The urine drainage device of the present invention is both environmentally friendly and re-usable. It can be washed on a daily basis in warm to hot water either by hand or in a washing machine. Simple washing with or without a mild soap or detergent prevents the formation of biological or crystalline deposits on the device and helps reduce the risk of infection and/or skin irritation.

Other advantages and embodiments of the invention are set forth in part in the description which follows, and in part, will be obvious from this description, or may be learned from the practice of the invention.

### DESCRIPTION OF THE DRAWINGS

The design of the urine drainage device is explained in detail by drawings, showing:

FIG. 1 A urine drainage device of the present invention as seen from a perspective from below.

FIG. 2 A urine drainage device of the present invention as seen from a perspective from above.

FIG. 3 A urine drainage device of the present invention as seen from a lateral perspective.

FIG. 4 A cross-section view A-A of FIG. 1, respectively FIG. 2.

FIG. 5 The detail from FIG. 3 marked by circle B.

### DETAILED DESCRIPTION OF THE INVENTION

As embodied and broadly described herein, the present invention is directed to a novel system for managing uncontrolled urination (incontinence) in females. The present invention is particularly directed to washable and re-usable external devices which can be comfortably worn by female subjects and which avoid soiling of laundry, run a low risk of causing urinary infections or skin irritation and allow a female subject to remain mobile while wearing the device.

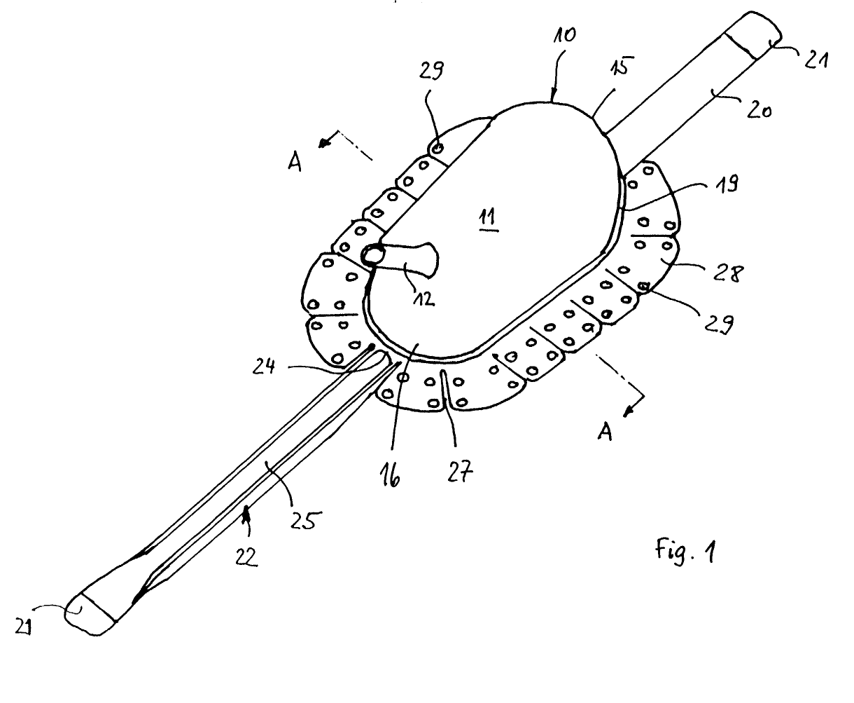

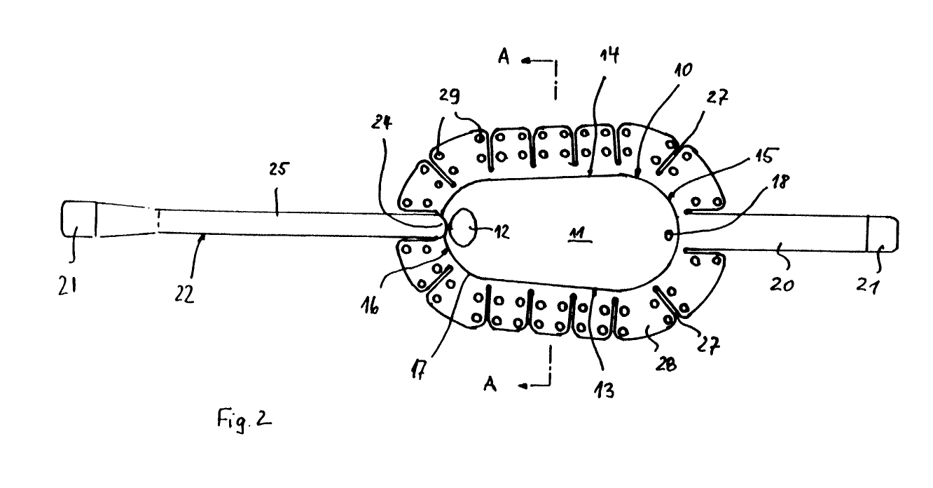

As depicted in FIGS. 1-5, the urine drainage device of the present invention comprises a hollow body 10. The hollow body 10 is preferably elongated and may be any appropriate shape to fit comfortably and securely around the vaginal area. The hollow body 10 may be made of any appropriate material which allows it to be comfortably worn by a female patient. Hollow body 10 is preferably made of a flexible, durable, and washable material such as, but not limited to, silicone-based materials, rubber or plastic.

In a preferred embodiment, hollow body 10 is shaped like a small bathtub, having a rounded off floor section 11 with a slightly larger, rounded off front wall 15 and a slightly smaller, rounded-off rear wall 16 opposite, connected by side walls 13 and 14 which are molded and rounded off into the floor section 11. Further in a preferred embodiment, side walls 13 and 14 converge slightly toward the smaller rear wall 16.

The device of the present invention further comprises a drainage spout 12. The drainage spout 12 may be located anywhere toward the distal end of the device where drainage of urine through drainage spout 12 is facilitated. Preferably, drainage spout 12 is located on the lowest point of hollow body 10. In a preferred embodiment, there is a funnel-shaped opening at the transition of smaller rear wall 16 forming a drain ending to which the drain spout 12 is attached. When the device is worn, its floor surface is thus tilted toward the drain which guarantees the complete emptying of the drainage device.

The urine drainage device of the present invention further comprises a rim 17 which is attached to hollow body 10 and is shaped to encompass the vagina. In one embodiment, hollow body 10 is molded at its top to form the rim 17. In a preferred embodiment, front and rear walls 15 and 16 and the two side walls 13 and 14, end at the same level in a rim 17.

The rim may be specifically designed to connect with an opening in specifically adapted tights or underwear which may be worn together with the device. In a preferred embodiment, below the rim 17 on the outside of the side walls 13 and 14 extends a support 19 to fit the outline of an opening in specifically designed tights or underwear.

The device further comprises at least one fastening body attached to the rim 17. The at least one fastening body may be any type of system of elastics bands, cloths, underwear shaped attachments, or any fastening system which holds the urine drainage device in place during wear. In one embodiment, the rim 17 extends into a lateral, outward-facing flange which forms part of the at least one fastening body. The flange may be molded from the same material as hollow body 10. The flange preferably covers the entire circumference of the rim and is designed to provide a seal against both thighs when worm. The flange is preferably designed to provide a watertight seal for the urine drainage device.

In a further embodiment, a flange may be split by basically vertical incisions towards the rim 17 into separate flange elements 28. The flange elements 28 may cover the entire circumference of the rim 17 with the exception of the centers of the front and rear walls 15 and 16. The flange elements 28 provide an improved molding to the skin and body shape of the wearer beneath the crotch curvature while avoiding overlap between segments which could cause some discomfort. In a preferred embodiment, each flange element comprises a plurality of holes 29. The holes reduce skin contact while simultaneously improving adhesion and permitting ventilation.

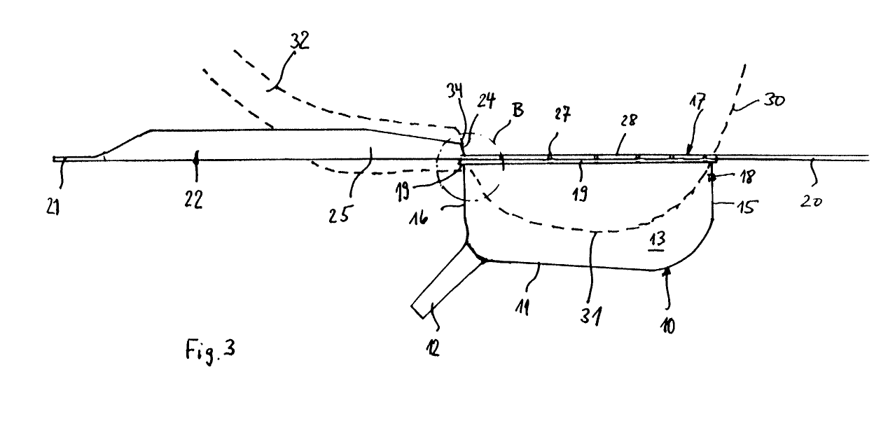

Further in a preferred embodiment, flaps 20 and 22 are molded onto hollow body 10 and separated by incisions 27 from their neighboring flange elements 28. As depicted in FIG. 3, band-shaped flap 20 is flexibly molded to shape itself comfortably to the pelvic bone 30. Flap 20 may protrude up from rim 17 to elevate the hollow body 10 above the skin. Being thus elevated reduces skin irritation by avoiding urine contamination in the region of the vaginal dam.

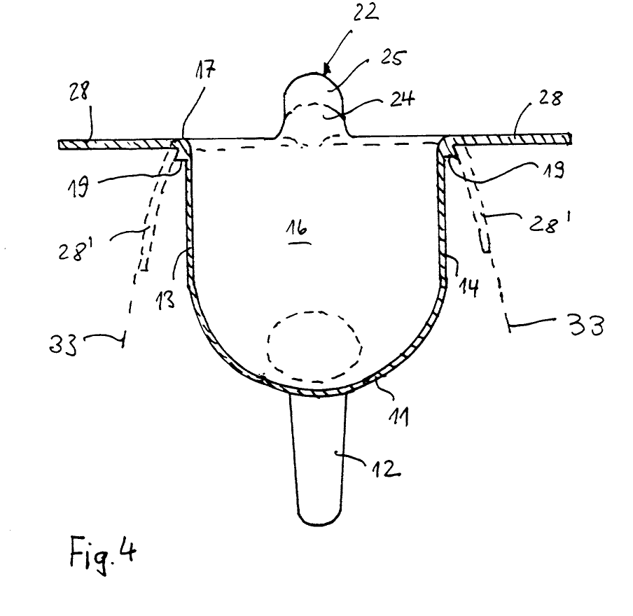

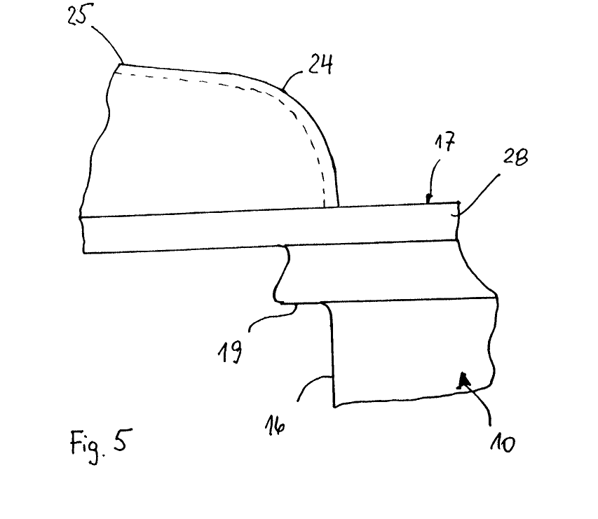

In a preferred embodiment, flap 22 protrudes from the smaller rear wall 16, as seen in FIGS. 4 and 5, and consists of an upward-pointing first section 24 which goes over into a second section 25, consisting of a three-walled tunnel profile, whose open side faces drain spout 12. The first section 24 is formed by and incorporates the body-facing wall of the tunnel profile in section 25. As depicted in FIG. 3, section 24 of flap 22 takes a sealing position against the vaginal dam 34 of the wearer while the second section 25 with its tunnel profile fits into anal crevice 32. The shape of a tunnel profile results in a good fit in the anal crevice. Flaps 20 and 22 are preferably made of the same material as hollow body 10, and comprise at least one fastening element 21 at their far ends. The at least one fastening element 21 may be any system of snaps, buttons, hook and loop fastening material (such as VELCRO), or any other appropriate system or combination thereof

The vulva 31 as shown shaded in FIG. 3 is situated inside hollow body 10, whose floor section 11 is slightly tilted toward drain spout 12 situated at its lowest point when hollow body 10 is positioned on the wearer by flaps 20 and 22. In a preferred embodiment, at the larger front wall 15 where band-shaped flap 20 protrudes on the side of the pelvic bone, there is a centrally positioned ventilation opening 18 slightly below support 19 to ventilate the interior of hollow body 10. The distance of opening 18 to the support rim 19 is calculated to avoid both contact against pelvic bone 30 which would prevent its proper functioning when the urine drainage device is worn, and urine leakage through the height of opening's position. This opening 18 allows damp air to escape thereby reducing the risk of skin rashes, and improves the draining of urine into the drainage spout by relieving and vacuum formation within hollow tube 10.

The urine drainage device may be put on by a wearer by attaching the fastening body to some type of holder, like a belt, pant or skirt waistline, or any other appropriate object which facilitates holding the device in place. If the fastening body of the urine drainage device is designed to incorporate its own holder, the device may simply by put on and then adjusted to ensure a proper seal with the vagina.

In a preferred embodiment, the urine drainage device is attached to a hip belt by at least one fastening element 21 on each of flaps 20 and 22. The hip belt may be put on the wearer at the height of the 3rd or 4th loin vertebrae. The urine drainage device is held between the wearer's legs by the band-shaped flap 20 and guided on the side of the pelvic bone fastened to the hip belt by fastening device 21 of flap 20. The flange elements 28 position themselves as drawn shaded as Position 28 in FIG. 4 to lie next to each other with a sealing function against the skin 33. The upward facing first section 24 of the tunnel-shaped flap 22 facing the vaginal dam lies in a sealing capacity between neighboring flange elements 28 against the vaginal dam 34, while the second section 25 with the tunnel-shaped profile extends into anal crevice 32 and, being flexible, adapts its shape to the width of the anal crevice.

Band-shaped flap 20 rests against pelvic bone 30, while the vulva 31 with the urine exit is situated inside the space of hollow body 10, whose rim forms a seal against the skin. This way, urine released into the hollow body 10 can drain easily through the drainage spout 12.

The urine drainage device of the present invention may be attached, via the drainage spout 12, to a urine collector, such as a bladder bag, preferably worn on the leg. Drainage spout 12 may be attached directly to a urine collector or it may be attached through a flexible tube.

The measurements of the urine drainage device correspond to the average measurements of the corresponding body area of the wearer. Larger or smaller sizes can be produced for which fastening elements 21 can easily be lengthened or shortened as required. The urine drainage device can be molded in one piece (i.e., integrally molded) from the same flexible material, preferably silicone-containing materials. Silicone is a very flexible and elastic material, very skin-friendly, unaffected by contact with body fluids, and easily and completely cleaned by hot water.

Other embodiments and uses of the invention will be apparent to those skilled in the art from consideration of the specification and practice of the invention disclosed herein. The specification and examples should be considered exemplary only with the true scope and spirit of the invention indicated by the following claims. As will be easily understood by those of ordinary skill in the art, variations and modifications of each of the disclosed embodiments can be easily made within the scope of this invention as defined by the following claims.

What is claimed is:

1. A device for the drainage of urine release in incontinent females, the device comprising:
(a) a hollow body;
(b) a drainage spout;
(c) a rim; and
(d) a fastening body;
wherein the device is worn externally of the female's body.

2. The device of claim 1 wherein the hollow body comprises:
(a) a floor section;
(b) two side walls;
(c) a front wall; and
(d) a rear wall;
wherein the two side walls, the front wall, and the rear wall are integrally connected with the floor section.

3. The device of claim 2 wherein the fastening body comprises a flange such that the rim is shaped to encompass the female's vaginal area and the flange protrudes outwardly from the rim.

4. The device of claim 3, further wherein the flange has a plurality of holes formed therein.

5. The device of claim 4, further wherein the flange is separated into a plurality of flange elements.

6. The device of claim 3 further wherein the fastening body comprises:
(a) a flap attached to the front wall; and
(b) a flap attached to the rear wall; each flap having a distal end comprising a fastening element.

7. The device of claim 6, wherein the fastening element is selected from the group consisting of snaps, buttons, and hook-and-loop fastening material.

8. The device of claim 2 wherein the front wall has at least one opening to ventilate the hollow body, said at least one opening being situated sufficiently below the rim to avoid sealing contact with a pelvic bone, and being sufficiently above the drainage spout to prevent leakage.

8. The device of claim 1 further comprising a support molded to the rim, which support is shaped so as to match an outline of an opening in tights or underwear which the female may choose to wear with the device.

9. The device of claim 1, further wherein the hollow body, the drainage spout, the rim, and the fastening body are integrally molded.

10. The device of claim 1, further wherein said device is made of a silicone based material.

11. A method for managing uncontrolled urine release in a female, comprising:
obtaining the device of claim 1
securing said device to the female's vaginal area.

12. The method of claim 11 further comprising attaching the drainage spout of the device to a urine collector.

13. The method of claim 12 further comprising securing the urine collector around a leg of the female.

14. The method of claim 12 wherein the step of attaching comprises attaching the drainage spout of the device to a bladder bag.

15. A method for managing uncontrolled urine release in a female, comprising:
obtaining the device of claim 6
securing said device to the female's vaginal area.

16. The method of claim 15 further comprising securing at least one fastening element of the device to a hip belt.

17. The method of claim 15 further comprising attaching the drainage spout of the device to a urine collector.

18. The method of claim 17 further comprising securing the urine collector around a leg of the female.

19. The method of claim 15 wherein the step of attaching comprises attaching the drainage spout of the device to a bladder bag.
